(19) United States
(12) Patent Application Publication
(10) Pub. No.: US 2025/0288779 A1
(43) Pub. Date: Sep. 18, 2025

(54) MEDICAL DEVICE AND METHOD OF MANUFACTURING MEDICAL DEVICE

(71) Applicant: ASAHI INTECC CO., LTD., Seto-shi (JP)

(72) Inventors: Aoi OKAMURA, Seto-shi (JP); Kenta TSUGE, Seto-shi (JP); Narumi NAGAO, Seto-shi (JP); Sho TSUCHIYA, Seto-shi (JP)

(73) Assignee: ASAHI INTECC CO., LTD., Seto-shi (JP)

(21) Appl. No.: 19/220,461

(22) Filed: May 28, 2025

Related U.S. Application Data
(63) Continuation of application No. PCT/JP2022/044154, filed on Nov. 30, 2022.

(51) Int. Cl. A61M 25/09

(52) U.S. Cl. CPC A61M 25/09; A61M 2025/09083; A61M 2025/09108; A61M 2205/0238; A61M 2207/00

(57) ABSTRACT

A medical device includes an outer layer coil having a first site that includes a section in which a coil pitch is a first pitch and a second site that includes the section in which the coil pitch is a second pitch that is wider than the first pitch, and a resin coating that covers the outer layer coil, a representative outer diameter of the resin coating covering an outer periphery of the second site being larger than the representative outer diameter of the resin coating covering the outer periphery of the first site.

### CROSS REFERENCE TO RELATED APPLICATIONS

This is a Continuation of PCT/JP2022/044154 filed on Nov. 30, 2022. The disclosure of the prior application is hereby incorporated by reference herein in its entirety.

### TECHNICAL FIELD

The disclosure relates to a medical device such as a guidewire or a catheter.

### BACKGROUND

A guidewire can be used for guiding a catheter to a desired position inside the body. Patent literature 1 describes a guidewire including a resin layer that covers the outer periphery of a coil.

### CITATION LIST

### Patent Literature

Patent Literature 1: JP 2022-152493 A

### SUMMARY

### Technical Problem

When a conventional guidewire is used inside the body, such as inside the blood vessel, the guidewire sometimes retracts in the opposite direction to its advancing direction due to resistance from the blood vessel wall or the like. This issue is not unique to a guidewire, but is also common to a medical device such as a catheter used inside the body.

An object of the disclosure is to provide a medical device that can prevent the occurrence of retraction when the medical device encounters resistance from the blood vessel wall or the like.

### Solution to Problem

The disclosure has been made to solve at least part of the above-mentioned problems, and can be achieved in the following aspects.

(1) One aspect of the disclosed embodiments is a medical device including: an outer layer coil having a first site that includes a section in which a coil pitch is a first pitch and a second site that includes the section in which the coil pitch is a second pitch, and the second pitch is wider than the first pitch; and a resin coating that covers the outer layer coil, a representative outer diameter of the resin coating covering an outer periphery of the second site being larger than a representative outer diameter of the resin coating covering an outer periphery of the first site.

According to this configuration, the representative outer diameter of the resin coating covering the outer periphery of the second site is larger than the representative outer diameter of the resin coating covering the outer periphery of the first site, thereby making it possible to prevent retraction of the medical device when the medical device encounters resistance from the blood vessel wall or the like.

### BRIEF DESCRIPTION OF DRAWINGS

FIG. 1 is an explanatory diagram illustrating an example of an overall configuration of a medical device of a first embodiment.

FIG. 2 is an explanatory diagram illustrating an example of a cross section taken along a line A-A in FIG. 1.

FIG. 3 is an explanatory diagram illustrating an example of a longitudinal section of a tip portion of the medical device.

FIG. 4 is an explanatory diagram illustrating an example of an enlarged view of a resin coating.

FIG. 5 is an explanatory diagram illustrating an example of a method for forming the medical device.

FIG. 6 is an explanatory diagram illustrating an example of a method for forming the resin coating.

FIG. 7 is an explanatory diagram illustrating an example of an enlarged view of the resin coating of the medical device of a second embodiment.

FIG. 8 is an explanatory diagram illustrating an example of an enlarged view of the resin coating of the medical device of a third embodiment.

FIG. 9 is an explanatory diagram illustrating an example of an enlarged view of the resin coating of the medical device of a fourth embodiment.

FIG. 10 is an explanatory diagram illustrating an example of an enlarged view of the resin coating of the medical device of a fifth embodiment.

FIG. 11 is an explanatory diagram illustrating an example of an enlarged view of the resin coating of the medical device of a sixth embodiment.

FIG. 12 is an explanatory diagram illustrating an example of a longitudinal section of the tip portion of the medical device of a seventh embodiment.

### DETAILED DESCRIPTION OF EMBODIMENTS

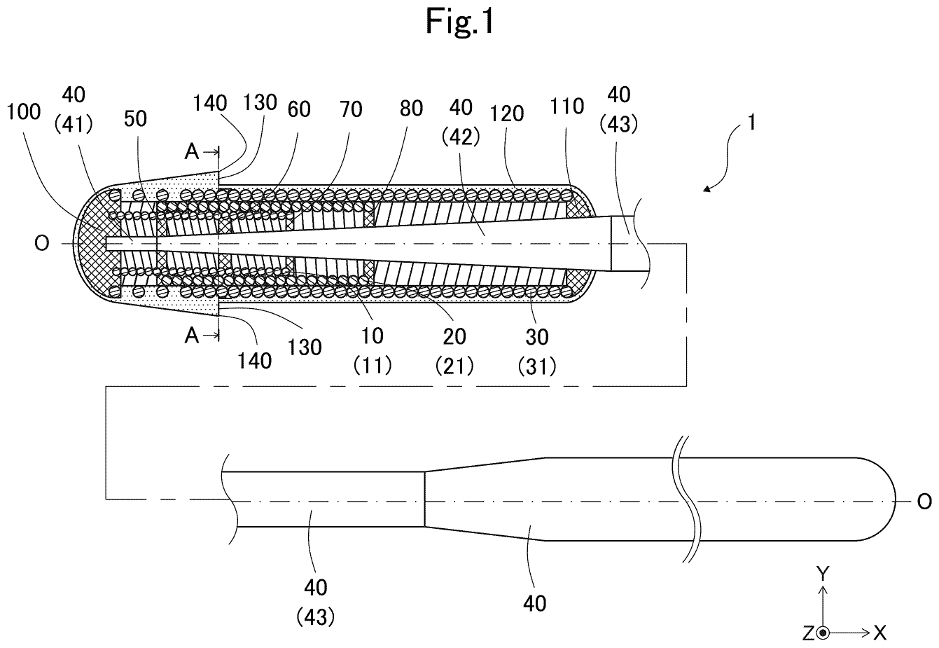

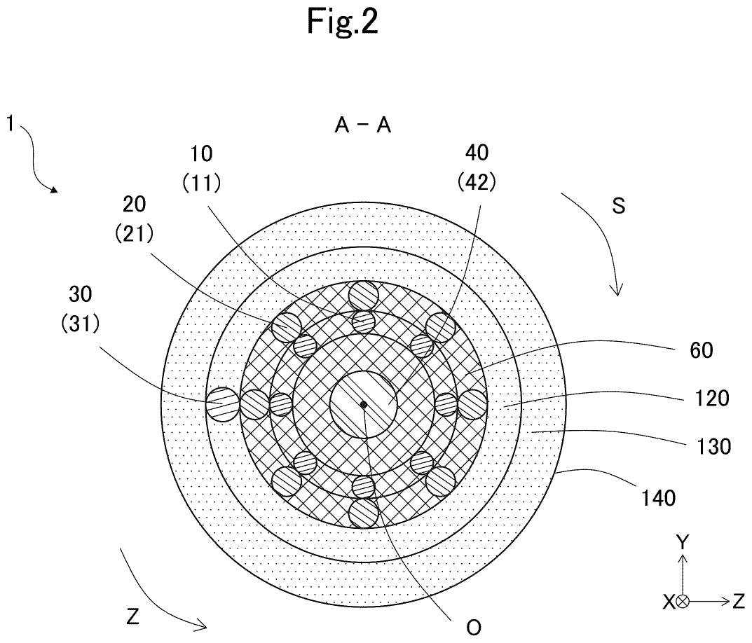

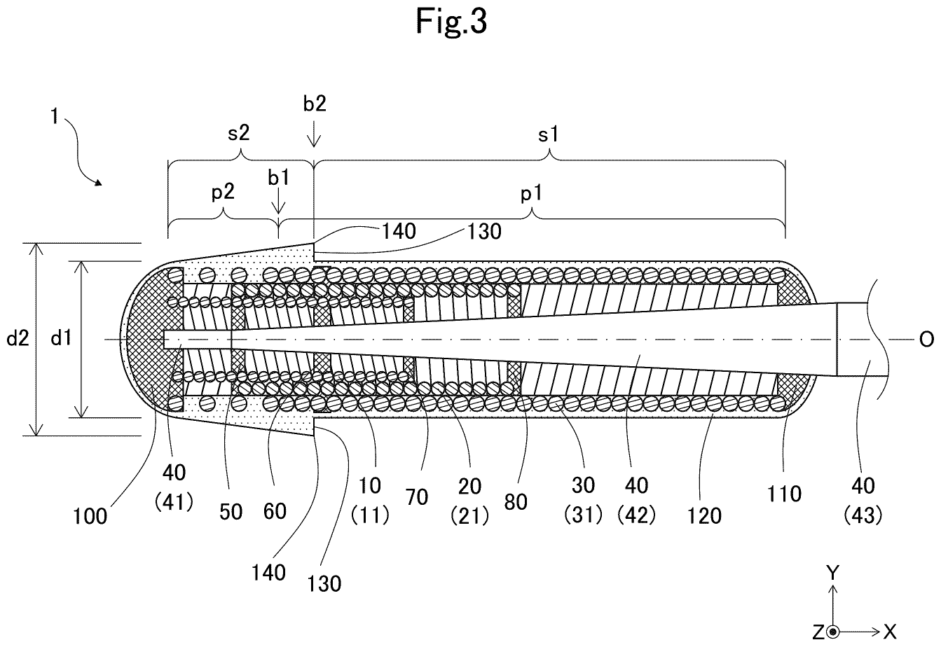

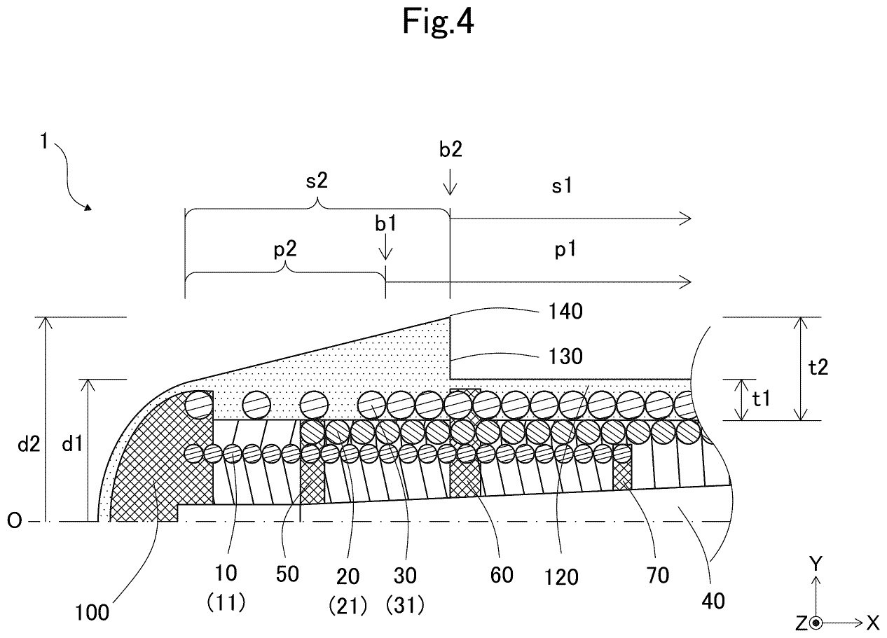

FIG. 1 is an explanatory diagram illustrating an example of the overall configuration of a medical device 1 of a first embodiment. FIG. 2 is an explanatory diagram illustrating an example of a cross section taken along a line A-A in FIG. 1. The medical device 1 of the first embodiment is described with reference to FIG. 1 to FIG. 6. Here, as an example, the medical device 1 is described as a guidewire used for inserting medical equipment such as a catheter into a blood vessel or the like. The medical device 1 includes a first coil body 10, a second coil body 20, a third coil body 30, a core shaft 40, a first fixing portion 50, a second fixing portion 60, a third fixing portion 70, a fourth fixing portion 80, a tip side fixing portion 100, and a base side fixing portion 110. FIG. 1, FIG. 3, and FIG. 4 are diagrams illustrating examples of longitudinal sections of members constituting the medical device 1. However, the core shaft 40 is illustrated in outline rather than in the longitudinal section. Note that, in the following examples, the medical device 1 is described as a device that is used in the blood vessel. However, the medical device 1 can be inserted and used in a body lumen, such as the one in a lymphatic system, a biliary system, a urinary system, a respiratory system, a digestive system, a secretory gland, or a reproductive organ, in addition to the vascular system.

In FIG. 1, an axis passing through the center of the medical device 1 is represented by an axis O (dotted line). In an example in FIG. 1, the axis O coincides with the axis passing through the centers of the first to third coil bodies (10, 20, 30) and the core shaft 40. Note that the axis O may be different from the central axis of each of the above-mentioned constituent members. FIG. 1 illustrates XYZ axes which are mutually orthogonal. The X axis corresponds to an axial direction of the medical device 1, the Y axis corresponds to a height direction of the medical device 1, and the Z axis corresponds to a width direction of the medical device 1. The left side of FIG. 1 (−X axis direction) is referred to as a “tip side” of the medical device 1 and each of the constituent members, and the right side of FIG. 1 (+X axis direction) is referred to as a “base side” of the medical device 1 and each of the constituent members. Further, of the two ends of the medical device 1 and each of the constituent members in the axial direction (X axis direction), the one end located on the tip side is referred to as a “tip end”, and the other end located on the base side is referred to as a “base end”. Further, the tip end and its vicinity are referred to as a “tip portion”, and the base end and its vicinity are referred to as a “base portion”. The tip side is inserted into the body, and the base side is operated by an operator such as a doctor. These points are common to FIG. 1 and subsequent drawings.

### <Core Shaft 40>

As illustrated in FIG. 1, the core shaft 40 has an elongated outer shape extending along the axis O. From the tip end to the base end, the core shaft 40 includes a straight portion 41, a tapered portion 42, and a large diameter portion 43.

The straight portion 41 is disposed at the most tip side of the core shaft 40. The straight portion 41 has an elongated shape extending coaxially with the axis O of the medical device 1. The outer diameter of the straight portion 41 is substantially constant along the axis O. The tip end of the straight portion 41 is fixed to the first coil body 10 and the third coil body 30 by a tip side fixing portion 100. The tapered portion 42 is connected to the base end of the straight portion 41, and the large diameter portion 43 is connected to the base end of the tapered portion 42.

The straight portion 41 is a member that facilitates shaping when an operator gives a curved shape to the tip portion of the medical device 1, and is also called a “ribbon”. The straight portion 41 may have a flattened cross section to facilitate the operator to give the curved shape. For example, although not illustrated, the straight portion 41 may have a cross-sectional shape in which the maximum length in the Y-axis direction is different from the maximum length in the Z-axis direction in the cross section. Specifically, the cross-sectional shape of the straight portion 41 may be a rectangle in which the maximum length in the Z-axis direction is longer than the maximum length in the Y-axis direction, or an ellipse.

The tapered portion 42 is disposed between the straight portion 41 and the large diameter portion 43. The tapered portion 42 is approximately a truncated cone-shaped portion whose outer diameter increases from the tip side to the base side in the axial direction of the core shaft 40. The straight portion 41 is connected to the tip end of the tapered portion 42, and the large diameter portion 43 is connected to the base end of the tapered portion 42. An example of the cross section of the tapered portion 42 is illustrated in FIG. 2. The cross section of the tapered portion 42 is circular.

The large diameter portion 43 is disposed on the base side of the core shaft 40. The large diameter portion 43 is approximately a cylindrically shaped portion having a substantially constant outer diameter from the base end to the tip end. The outer diameter of the large diameter portion 43 is the same as the largest diameter of the tapered portion 42. Note that, in the present embodiment, the term “same” means substantially the same, allowing a difference due to a manufacturing error or the like. The tip end of the large diameter portion 43 is connected to the tapered portion 42.

A material of the core shaft 40 is not particularly limited. However, for example, stainless steel (SUS302, SUS304, SUS316, etc.), a superelastic alloy (also referred to as “pseudoelastic alloy”) such as a Ni—Ti alloy, a piano wire, a nickel-chromium-based alloy, a cobalt alloy, platinum, gold, tungsten, or the like can be used. The straight portion 41, the tapered portion 42, and the large diameter portion 43 may be made of the same material, or each may be made of a different material.

### <First Coil Body 10>

The first coil body 10, the second coil body 20, and the third coil body 30 are disposed on the outer periphery of the core shaft 40 in this order from the radially inner side to the radially outer side of the medical device 1. As illustrated in FIG. 2, the first coil body 10 is a coil body formed by eight wires 11 wound in a spiral shape in the axial direction of the medical device 1. As illustrated in FIG. 1, the first coil body 10 is disposed so as to surround the tip portion of the core shaft 40. Specifically, the first coil body 10 is disposed so as to surround the straight portion 41 of the core shaft 40 and a part of the tip side of the tapered portion 42. The tip end of the first coil body 10 is fixed to the core shaft 40 and the third coil body 30 by the tip side fixing portion 100. The base end of the first coil body 10 is fixed to the core shaft 40 by the third fixing portion 70.

The first coil body 10 is wound in a first winding direction S as illustrated in FIG. 2. The first coil body 10 is wound in a first winding direction S as illustrated in FIG. 2. The first winding direction S is a clockwise rotation direction around the axis O passing through the center of the medical device 1, from a viewpoint of observing the medical device 1 from the tip side to the base side (that is, in the +X axis direction).

### <Second Coil Body 20>

The second coil body 20 is a coil body formed by eight wires 21 wound in a spiral shape in the axial direction of the medical device 1. The second coil body 20 is disposed radially outward of the first coil body 10 in the medical device 1 so as to surround a part of the tapered portion 42 and a part of the first coil body 10. In the axial direction of the medical device 1, the tip end of the second coil body 20 is located between the tip end and base end of the first coil body 10. The tip end of the second coil body 20 is fixed to the first coil body 10 and the core shaft 40 by the first fixing portion 50. Further, in the axial direction of the medical device 1, the base end of the second coil body 20 is located closer to the base side than the base end of the first coil body 10. An intermediate part of the second coil body 20 located between the tip end and base end of the second coil body 20 is fixed to the first coil body 10, the third coil body 30, and the core shaft 40 by the second fixing portion 60. The base end of the second coil body 20 is fixed to the core shaft 40 by the fourth fixing portion 80. The second coil body 20 is wound in the first winding direction S as illustrated in FIG. 2. That is, the winding directions of the first coil body 10 and the second coil body 20 are the same.

### <Third Coil Body 30>

The third coil body 30 is a coil body formed by one wire 31 wound in a spiral shape in the axial direction of the medical device 1. The third coil body 30 is disposed radially outward of the second coil body 20 in the medical device 1 so as to surround a part of the core shaft 40 (in the present embodiment, the straight portion 41 and a part of the tip side of the tapered portion 42), the first coil body 10, and the second coil body 20. In the axial direction of the medical device 1, the tip end of the third coil body 30 is at the same position as the tip end of the first coil body 10. The tip end of the third coil body 30 is fixed to the core shaft 40 and the first coil body 10 by the tip side fixing portion 100. Further, in the axial direction of the medical device 1, the base end of the third coil body 30 is located closer to the base side than the base end of the second coil body 20. The base end of the third coil body 30 is fixed to the core shaft 40 by the base side fixing portion 110. A pitch of the wire 31 varies depending on the axial position of the third coil body 30. The pitch of the wire 31 is described below. Further, in the present embodiment, the third coil body 30 is divided into a first site s1 (FIG. 3) and a second site s2 (FIG. 3) depending on the size of the pitch of the wire 31 of the third coil body 30, as is described in detail below.

The third coil body 30 is wound in a second winding direction Z as illustrated in FIG. 2. The third coil body 30 is wound in a second winding direction Z as illustrated in FIG. 2. The second winding direction Z is a counterclockwise rotation direction around the axis O passing through the center of the medical device 1, from a viewpoint of observing the medical device 1 from the tip side to the base side (that is, in the +X axis direction).

The configurations of the first to third coil bodies (10, 20, 30) are not limited to the above, and any embodiments can be adopted. For example, the number of wires (11, 21, 31) constituting the first to third coil bodies (10, 20, 30) can be freely determined. The first coil body 10 and the second coil body 20 are not limited to a multi-stranded coil, and may be a single stranded coil formed by winding one wire into a single strand, a single stranded twisted coil formed by winding a twisted wire obtained by twisting multiple wires together into a single strand, or a multi-stranded twisted coil formed by using multiple twisted wires obtained by twisting multiple wires together and winding the twisted wires into multiple strands.

Materials of the first to third coil bodies (10, 20, 30) are not particularly limited. However, the first to third coil bodies can be formed of, for example, a stainless steel alloy such as SUS304 or SUS316, a superelastic alloy such as a Ni—Ti alloy, a piano wire, a radiolucent alloy such as a nickel-chromium-based alloy or a cobalt alloy, gold, platinum, tungsten, or a radiopaque alloy such as an alloy containing these elements (e.g., a platinum-nickel alloy). The first to third coil bodies (10, 20, 30) may be formed of the same material or different materials.

### <Fixing Portion>

The core shaft 40, the first coil body 10, the second coil body 20, and the third coil body 30 that constitute the medical device 1 are fixed by multiple fixing portions. The medical device 1 includes, in order from the tip side of the medical device 1, the tip side fixing portion 100, the first fixing portion 50, the second fixing portion 60, the third fixing portion 70, the fourth fixing portion 80, and the base side fixing portion 110.

The tip side fixing portion 100 is disposed at the tip end of the third coil body 30 and fixes the tip end of the third coil body 30, the tip end of the core shaft 40, and the tip end of the first coil body 10. The base side fixing portion 110 is disposed at the base end of the third coil body 30 and fixes the base end of the third coil body 30 and a part of the tapered portion 42. The first fixing portion 50 is disposed at the tip end of the second coil body 20 and fixes the tip end of the second coil body 20 and a part of the first coil body 10. The second fixing portion 60 is disposed at an intermediate part of the second coil body 20 and fixes the first coil body 10, the second coil body 20, the third coil body 30, and the core shaft 40. The third fixing portion 70 is disposed at the base end of the first coil body 10 and fixes the base end of the first coil body 10 and a part of the tapered portion 42. The fourth fixing portion 80 is disposed at the base end of the second coil body 20 and fixes the base end of the second coil body 20 and a part of the tapered portion 42.

The tip side fixing portion 100, the base side fixing portion 110, the first fixing portion 50, the second fixing portion 60, the third fixing portion 70, and the fourth fixing portion 80 can be formed by any bonding agent, for example, metal solder such as silver solder, gold solder, zinc, an Sn—Ag alloy, or an Au—Sn alloy, or an adhesive such as an epoxy adhesive. The tip side fixing portion 100, the base side fixing portion 110, the first fixing portion 50, the second fixing portion 60, the third fixing portion 70, and the fourth fixing portion 80 may be formed by using the same bonding agent or may be formed by using different bonding agents. Note that the tip side fixing portion 100, the base side fixing portion 110, the first fixing portion 50, the second fixing portion 60, the third fixing portion 70, and the fourth fixing portion 80 may be formed by laser welding.

### <Resin Coating 120>

The resin coating 120 is a thin resin film that covers the outer periphery of the third coil body 30. The resin coating 120 includes a part whose thickness changes in the axial direction of the resin coating 120, and the outer diameter also changes due to the change in thickness. The resin coating 120 includes a step portion 130 formed on the outer periphery at the part where the outer diameter of the resin coating 120 changes. A part of the resin coating 120 with the largest outer diameter is referred to as a “maximum outer diameter portion 140”. In the present embodiment, the resin coating 120 covers the outer periphery of the third coil body 30. However, the resin coating 120 may also cover the outer periphery of the tapered portion 42 of the core shaft 40 or the outer periphery of the large diameter portion 43. Details of the resin coating 120 are described below.

A material of the resin coating 120 is not particularly limited. However, the resin coating 120 can be formed of, for example, a hydrophobic resin material, a hydrophilic resin material, or a mixture thereof. Examples of the hydrophobic resin material that can be used include a silicone resin, polyurethane, polyethylene, polyvinyl chloride, polyester, polypropylene, polyamide, polystyrene, polyolefin elastomer, polyester elastomer, polyamide elastomer, and polyurethane elastomer. Examples of the hydrophilic resin material that can be used include a starch-based material such as carboxymethyl starch, a cellulose-based material such as carboxymethyl cellulose, a polysaccharide such as alginic acid, chitin, chitosan, or hyaluronic acid, a natural water-soluble polymeric material such as gelatin, and a synthetic water-soluble polymeric material such as polyvinyl alcohol, polyethylene oxide, polyethylene glycol, polypropylene glycol, polyvinyl pyrrolidone, or water-soluble nylon. The resin coating 120 may be formed of different materials depending on the axial position of the resin coating 120.

### <Pitch of Wire 31 of Third Coil Body 30>

FIG. 3 is an explanatory diagram illustrating an example of a longitudinal section of a tip portion of the medical device 1. As described above, the pitch (coil pitch) of the wire 31 of the third coil body 30 varies depending on the axial position of the third coil body 30. A part of the coil pitch of the third coil body 30 is set to a first pitch, and the other part of the coil pitch is set to a second pitch wider than the first pitch. In FIG. 3, in the third coil body 30, a section set to the first pitch is represented by p1 and referred to as a “first pitch section p1” below. In FIG. 3, in the third coil body 30, a section set to the second pitch is represented by p2 and referred to as a “second pitch section p2” below. In the present embodiment, the first pitch section p1 is disposed closer to the base side than the second pitch section p2.

The size of the first pitch is not particularly limited, and the pitch may be, for example, sized such that coils of the spirally wound wire 31 touch to each other, or such that a gap is formed between coils of the wire 31. The size of the second pitch is not particularly limited and can be, for example, about 1.01 times to about 100 times, preferably 1.01 times to about 1.2 times, the first pitch.

In the axial direction of the third coil body 30, a switching position of the first pitch and the second pitch is referred to as a “boundary b1”. In other words, the boundary b1 is the boundary between the first pitch section p1 and the second pitch section p2. For example, if the first pitch gradually increases in size toward the tip side of the third coil body 30 and gradually changes to the second pitch, a specified section where the first pitch transitions to the second pitch is the boundary b1.

<First Site s1 and Second Site s2>

In the present embodiment, for convenience of explanation, a specified range including the first pitch section p1 in the axial direction of the third coil body 30 is referred to as a “first site s1”. The first site s1 does not include the second pitch section p2. A specified range including the second pitch section p2 in the axial direction of the third coil body 30 is referred to as a “second site s2”. The second site s2 includes a part of the first pitch section p1. In this configuration, the first site s1 is a site of the first pitch section p1 excluding a part of the tip side of the first pitch section p1, and the second site s2 is a site including the second pitch section p2 and the part of the tip side of the first pitch section p1. Since the second pitch section p2 is disposed on the tip side of the third coil body 30, and the first pitch section p1 is disposed on the base side of the third coil body 30, the second site s2 is disposed on the tip side of the first site s1 in the axial direction of the third coil body 30. Hereinafter, the boundary between the first site s1 and the second site s2 is referred to as a “boundary b2”. As shown in a sixth embodiment described below, the second site s2 may be provided closer to the base side than the first site s1.

At the boundary b1 between the first site s1 and the second site s2, the second fixing portion 60 is formed to fix adjacent coils of the wire 31 of the third coil body 30. By fixing the adjacent coils of the wire 31, it becomes easy to change the pitch of the wire 31 at the boundary separated by the second fixing portion 60. As described above, the second fixing portion 60 fixes the core shaft 40 and the third coil body 30, and therefore fixes the relative position of the third coil body 30 to the core shaft 40 in the axial direction of the medical device 1. Further, providing the second fixing portion 60 makes it possible to use the second fixing portion 60 as a marker for the position where the thickness is changed when the resin coating 120 is formed on the surface of the third coil body 30.

### <Details of Resin Coating 120>

As described above, there are parts in which the thickness of the resin coating 120 varies depending on the axial position of the resin coating 120. In the present embodiment, the part of the resin coating 120 that covers the outer periphery of the first site s1 of the third coil body 30 has a straight shape with a substantially constant outer diameter in the axial direction of the resin coating 120. The part of the resin coating 120 that covers the second site s2 of the third coil body 30 has a tapered shape with an outer diameter that increases from the tip side to the base side of the resin coating 120. This makes it easy to insert the medical device 1 into a blood vessel or a catheter and advance it forward.

The outer diameter of the resin coating 120 varies depending on the axial position of the resin coating 120 as described above. Here, the outer diameter of the resin coating 120 covering the outer periphery of the first site s1 is referred to as an “outer diameter d1”, and the outer diameter of the resin coating 120 covering the outer periphery of the second site s2 is referred to as an “outer diameter d2”. Further, one value representative of the outer diameter d1 of the resin coating 120 covering the outer periphery of the first site s1 is referred to as a “representative outer diameter rd1”, and one value representative of the outer diameter d2 of the resin coating 120 covering the outer periphery of the second site s2 is referred to as a “representative outer diameter rd2”. The representative outer diameter rd1 may be the maximum value of the outer diameter d1 of the resin coating 120 covering the outer periphery of the first site s1, or may be the average value of the outer diameter d1. Further, the representative outer diameter rd2 may be the maximum value of the outer diameter d2 of the resin coating 120 covering the outer periphery of the second site s2, or may be the average value of the outer diameters d2. When the representative outer diameters (rd1, rd2) are the maximum values, the representative outer diameter rd1 is the maximum outer diameter of the resin coating 120 covering the outer periphery of the first site s1, and the representative outer diameter rd2 is the maximum outer diameter of the resin coating 120 covering the outer periphery of the second site s2.

When the representative outer diameters (rd1, rd2) are the average outer diameters, the average value of the outer diameter d1 is the average value when the outer diameter d1 of the resin coating 120 covering the outer periphery of the first site s1 is measured at 10 equally spaced points at any interval along the axial direction. The average value of the outer diameter d2 is the average value when the outer diameter d2 of the resin coating 120 covering the outer periphery of the second site s2 is measured at 10 equally spaced points at any interval along the axial direction. In the resin coating 120 of the present embodiment, the representative outer diameter rd2 of the resin coating 120 covering the outer periphery of the second site s2 is larger than the representative outer diameter rd1 of the resin coating 120 covering the outer periphery of the first site s1. That is, the resin coating 120 of the present embodiment satisfies at least one of the following (1) and (2).

(1) The maximum outer diameter of the resin coating 120 covering the outer periphery of the second site s2 is larger than the maximum outer diameter of the resin coating 120 covering the outer periphery of the first site s1.
(2) The average outer diameter of the resin coating 120 covering the outer periphery of the second site s2 is larger than the average outer diameter of the resin coating 120 covering the outer periphery of the first site s1.

A step 130 is formed on the outer periphery of the resin coating 120 due to the difference between the outer diameter d1 of the resin coating 120 covering the outer periphery of the first site s1 and the outer diameter d2 of the resin coating 120 covering the outer periphery of the second site s2. That is, the step 130 is formed in the part of the resin coating 120 where the outer diameter decreases from the size of the outer diameter d2 to the size of the outer diameter d1. The step 130 has a surface that is substantially perpendicular to the axial direction of the medical device 1.

A part of the second site s2 of the resin coating 120 that has the largest outer diameter d2 is referred to as a “maximum outer diameter portion 140”. The maximum outer diameter portion 140 has the largest outer diameter of the resin coating 120. The maximum outer diameter portion 140 is formed at a position closer to the base end than to the tip end of the second site s2. In the present embodiment, the position of the maximum outer diameter portion 140 in the axial direction of the medical device 1 is the same as the boundary b2 between the first site s1 and the second site s2. Further, in the present embodiment, the axial position of the maximum outer diameter portion 140 is the same as the axial position of the step portion 130.

FIG. 4 is an explanatory diagram illustrating an example of an enlarged view of the resin coating 120. A film thickness of the resin coating 120 varies depending on the axial position of the resin coating 120. Here, the film thickness of the resin coating 120 covering the outer periphery of the first site s1 is referred to as a “film thickness t1”, and the film thickness of the resin coating 120 covering the outer periphery of the second site s2 is referred to as a “film thickness t2”. Further, one value representative of the film thickness t1 of the resin coating 120 covering the outer periphery of the first site s1 is referred to as a “representative film thickness rt1”, and one value representative of the film thickness t2 of the resin coating 120 covering the outer periphery of the second site s2 is referred to as a “representative film thickness rt2”. The representative film thickness rt1 may be the maximum value of the film thickness t1 of the resin coating 120 covering the outer periphery of the first site s1, or may be the average value of the film thickness t1. Further, the representative film thickness rt2 may be the maximum value of the film thickness t2 of the resin coating 120 covering the outer periphery of the second site s2, or may be the average value of the film thicknesses t2. When the representative film thicknesses (rt1, rt2) are the maximum values, the representative film thickness rt1 is the maximum film thickness of the resin coating 120 covering the outer periphery of the first site s1, and the representative film thickness rt2 is the maximum film thickness of the resin coating 120 covering the outer periphery of the second site s2.

When the representative film thicknesses (rt1, rt2) are the average film thicknesses, the average value of the film thickness t1 is the average value when the film thickness t1 of the resin coating 120 covering the outer periphery of the first site s1 is measured at 10 equally spaced points at any interval along the axial direction. The average value of the film thickness t2 is the average value when the film thickness t2 of the resin coating 120 covering the outer periphery of the second site s2 is measured at 10 equally spaced points at any interval along the axial direction.

In the resin coating 120 of the present embodiment, the representative film thickness rt2 of the resin coating 120 covering the outer periphery of the second site s2 is larger than the representative film thickness rt1 of the resin coating 120 covering the outer periphery of the first site s1. That is, the resin coating 120 of the present embodiment satisfies at least one of the following (1) and (2).

(1) The maximum film thickness of the resin coating 120 covering the outer periphery of the second site s2 is larger than the maximum film thickness of the resin coating 120 covering the outer periphery of the first site s1.
(2) The average film thickness of the resin coating 120 covering the outer periphery of the second site s2 is larger than the average film thickness of the resin coating 120 covering the outer periphery of the first site s1.

### <Method for Forming Resin Coating 120>

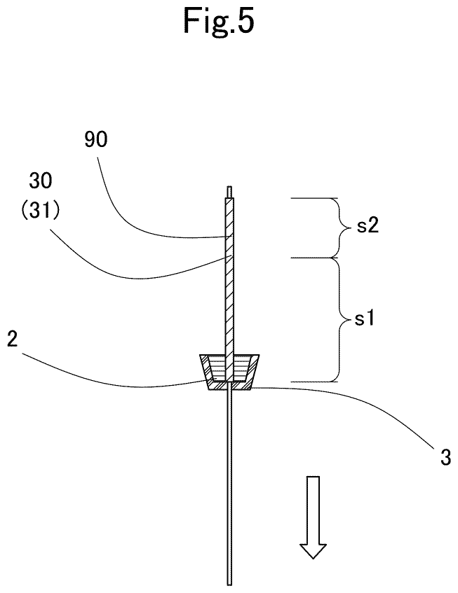

FIG. 5 is an explanatory diagram illustrating an example of a method for forming the medical device 1. An assembly 90 including the first to third coil bodies 10, 20, 30 and the core shaft 40 is prepared. The tip side of the assembly 90 is passed through a die 3, and the die 3 is filled with a resin 2, which is a material of the resin coating 120, and heated. The assembly 90 is moved in the direction of an arrow in FIG. 5. A thin film (resin coating 120) made of the resin 2 is formed on the outer periphery of the third coil body 30 of the assembly 90 passing through the die 3 along the axial direction of the third coil body 30.

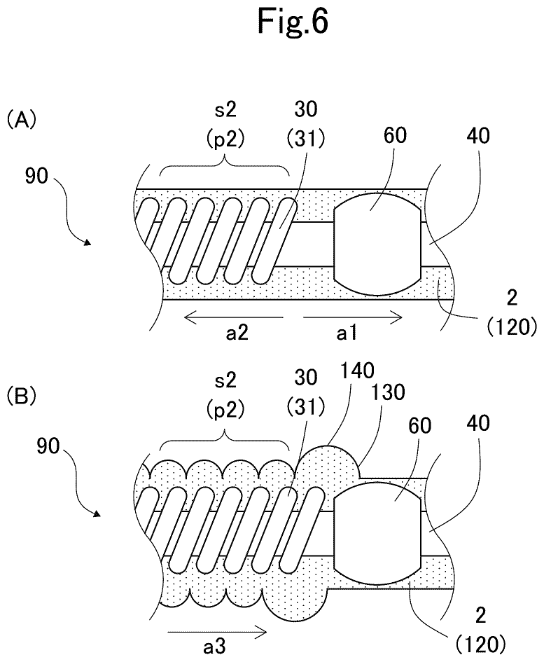

FIG. 6 is an explanatory diagram illustrating an example of a method for forming the resin coating 120. Note that, in FIG. 6, members such as the first coil body 10 and the second coil body 20 of the assembly 90 are omitted. (A) of FIG. 6 illustrates a step of applying the resin 2 to the outer periphery of the second site s2 of the third coil body 30. (B) of FIG. 6 illustrates a state in which the resin 2 is cooled and cured in the assembly 90. When the assembly 90 moves in the direction of an arrow a1 illustrated in (A) of FIG. 6, the resin 2 is applied to the outer periphery of the third coil body 30. At this time, the application of the resin 2 causes the wire 31 to move in the direction of an arrow a2 opposite to the moving direction of the assembly 90 (the direction of the arrow a1), and the resin 2 enters between the wire 31 and the second fixing portion 60 and into a gap between adjacent coils of the wire 31. Then, as illustrated in (B) of FIG. 6, the wire 31 moves in the direction of an arrow a3 illustrated in (B) of FIG. 6 due to its own elasticity to return to the position before the resin 2 was applied. At this time, the resin 2 that has entered between the wire 31 and the second fixing portion 60 and into the gap between the adjacent coils of the wire 31 is pushed and protrudes outward in the radial direction of the assembly 90. When the resin 2 is cooled, the resin 2 is cured in a protruding state to form the step portion 130, the maximum outer diameter portion 140, and the like of the resin coating 120. The medical device 1 is produced in this manner. Using the method described above as an example, the representative outer diameter rd2 of the resin coating 120 covering the outer periphery of the second site s2 can be formed to be larger than the representative outer diameter rd1 of the resin coating 120 covering the outer periphery of the first site s1.

According to the medical device 1 of the first embodiment described above, the representative outer diameter rd2 of the resin coating 120 covering the outer periphery of the second site s2 is larger than the representative outer diameter rd1 of the resin coating 120 covering the outer periphery of the first site s1. Since the representative outer diameter rd2 of the resin coating 120 covering the outer periphery of the second site s2 is larger, the distance between the outer periphery of the resin coating 120 and the inner periphery of medical equipment such as a catheter is reduced, and the area of contact between the resin coating 120 and the inner periphery of the medical equipment is increased. The frictional resistance between the medical device 1 and the medical equipment makes it possible to prevent retraction of the medical device 1 when the medical device 1 encounters resistance from the blood vessel wall or the like.

The second fixing portion 60 is formed at the boundary b2 between the first site s1 and the second site s2 to fix the coils of the wire 31 of the third coil body 30. This limits the movement of the wire 31 in the axial direction of the third coil body 30, making it possible to easily set the position of the first pitch section p1 and the position of the second pitch section p2.

The resin coating 120 includes the step portion 130. For example, when the medical device 1 is inserted into the medical equipment such as a catheter and used, the tip end of the medical device 1 may be disposed closer to the tip side than the tip end of the catheter. In such a state, when the medical device 1 is retracted in the opposite direction to the advancing direction, the step portion 130 contacts the tip end of the catheter or the like. This allows the step portion 130 to function as a stopper and prevent the medical device 1 from retracting. In particular, when the medical device 1 is inserted into the blood vessel of the lower limb of a human body, the medical device 1 may be first inserted into the blood vessel of one leg, and then passed through the common iliac artery and advanced to the other leg. If a part with a relatively low rigidity, such as the tapered portion 42 of the medical device 1, is disposed in the common iliac artery, the medical device 1 may retract in the blood vessel. Even in such a case, the step portion 130 can be caught by the tip end of the catheter or the like, making it possible to prevent the retraction of the medical device 1 when the medical device 1 encounters resistance from the blood vessel wall or the like.

The maximum outer diameter portion 140 is formed at a position closer to the base end than to the tip end of the second site s2. This makes it possible to keep the outer diameter of the tip portion of the medical device 1 small and to prevent the retraction of the medical device 1 when the medical device 1 encounters resistance from the blood vessel wall or the like.

The representative outer diameters (rd1, rd2) of the resin coating 120 are the maximum outer diameters of the resin coating 120. As a result, the maximum outer diameter of the resin coating 120 covering the outer periphery of the second site s2 is larger than the maximum outer diameter of the resin coating 120 covering the outer periphery of the first site s1. By reducing the distance between the outer periphery of the resin coating 120 and the inner periphery of the medical equipment such as a catheter, the area of contact between the resin coating 120 and the inner periphery of the medical equipment is increased. The frictional resistance between the medical device 1 and the medical equipment makes it possible to prevent the retraction of the medical device 1 when the medical device 1 encounters resistance from the blood vessel wall or the like.

The representative outer diameters (rd1, rd2) of the resin coating 120 are the average outer diameters of the resin coating 120. As a result, the average outer diameter of the resin coating 120 covering the outer periphery of the second site s2 becomes larger than the average outer diameter of the resin coating 120 covering the outer periphery of the first site s1. By reducing the distance between the outer periphery of the resin coating 120 and the inner periphery of the medical equipment such as a catheter, the area of contact between the resin coating 120 and the inner periphery of the medical equipment is increased. The frictional resistance between the medical device 1 and the medical equipment makes it possible to prevent the retraction of the medical device 1 when the medical device 1 encounters resistance from the blood vessel wall or the like.

In the resin coating 120, the representative film thickness rt2 of the resin coating 120 covering the outer periphery of the second site s2 is larger than the representative film thickness rt1 of the resin coating 120 covering the outer periphery of the first site s1. Since the representative film thickness rt2 of the resin coating 120 covering the outer periphery of the second site s2 is larger, the distance between the outer periphery of the resin coating 120 and the inner periphery of the medical equipment such as a catheter is reduced, and the area of contact between the resin coating 120 and the inner periphery of the medical equipment is increased. The frictional resistance between the medical device 1 and the medical equipment can prevent the retraction of the medical device 1. Further, the larger representative film thickness rt2 can prevent damage to the resin coating 120.

In the method for producing the medical device 1, the third coil body 30 including the first site s1 and the second site s2 is coated with the resin 2 such that the representative outer diameter of the resin coating 120 covering the outer periphery of the second site s2 is made larger than the representative outer diameter of the resin coating 120 covering the outer periphery of the first site s1. This makes it possible to easily produce the medical device 1 in which the representative outer diameter of the resin coating 120 covering the outer periphery of the second site s2 is made larger than the representative outer diameter of the resin coating 120 covering the outer periphery of the first site s1.

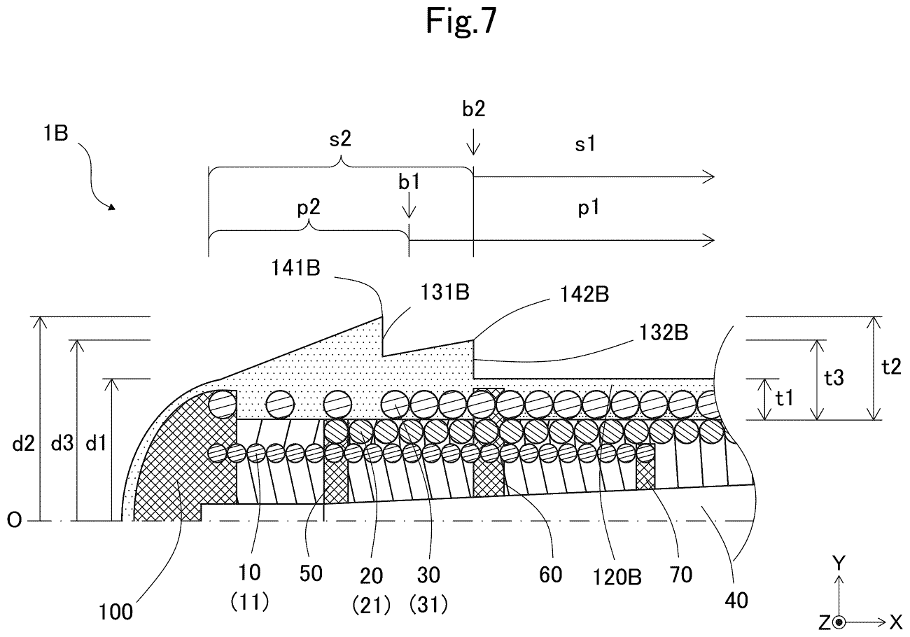

FIG. 7 is an explanatory diagram illustrating an example of an enlarged view of a resin coating 120B of a medical device 1B of a second embodiment. The medical device 1B is different from the medical device 1 of the first embodiment in that the resin coating 120B includes two step portions (131B, 132B) and two maximum outer diameter portions (141B, 142B). The description of the configuration of the medical device 1B that is common to the medical device 1 of the first embodiment is omitted.

The resin coating 120B includes the step portion 131B on the tip side in the axial direction of the resin coating 120B, and the step portion 132B on the base side. The step portion 131B is disposed closer to the tip side than the boundary b2 between the first site s1 and the second site s2. The axial position of the step portion 132B is the same as the axial position of the boundary b2. The resin coating 120B includes the maximum outer diameter portion 141B on the tip side in the axial direction of the resin coating 120B, and the maximum outer diameter portion 142B on the base side. The maximum outer diameter portion 141B is disposed closer to the tip side than the boundary b2 between the first site s1 and the second site s2. The axial position of the maximum outer diameter portion 142B is the same as the axial position of the boundary b2. The relationship between the outer diameters (d2, d3, d1) of the maximum outer diameter portion 141B, the maximum outer diameter portion 142B, and the resin coating 120 covering the outer periphery of the first site s1 is as follows:

outer diameter d2>outer diameter d3>outer diameter d1.

The relationship between the film thicknesses (t2, t3, t1) of the maximum outer diameter portion 141B, the maximum outer diameter portion 142B, and the resin coating 120 covering the outer periphery of the first site s1 is as follows:

film thickness t2>film thickness t3>film thickness t1.

With the medical device 1B of the second embodiment described above, the distance between the outer periphery of the resin coating 120B and the inner periphery of the medical equipment such as a catheter is reduced, and the area of contact between the resin coating 120B and the inner periphery of the medical equipment is increased. Thus, the frictional resistance between the medical device 1B and the medical equipment can prevent the retraction of the medical device 1B when the medical device 1B encounters resistance from the blood vessel wall or the like.

### Third Embodiment

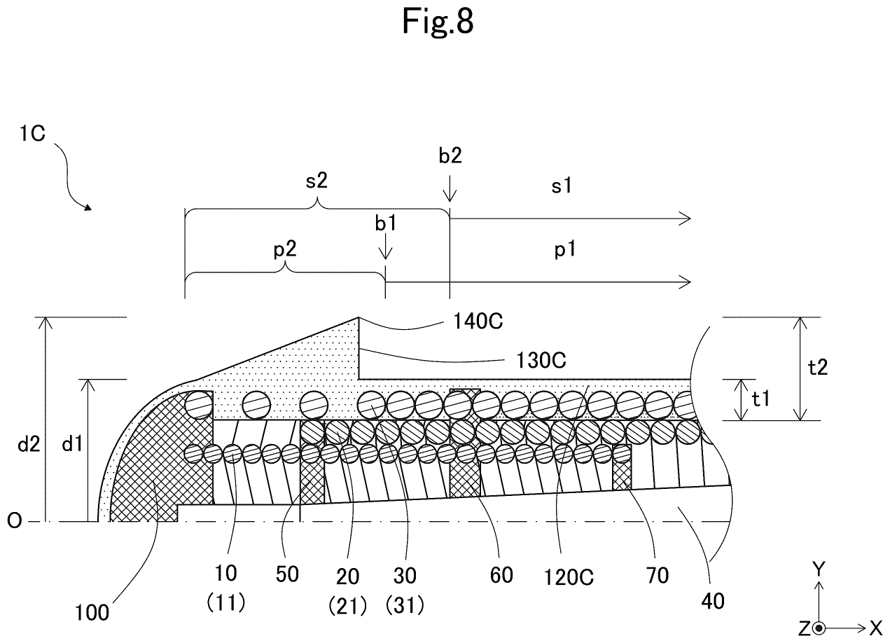

FIG. 8 is an explanatory diagram illustrating an example of an enlarged view of a resin coating 120C of a medical device 1C of a third embodiment. The medical device 1C is different from the medical device 1 of the first embodiment in that the axial position of the step portion 130C and the axial position of the maximum outer diameter portion 140C are located closer to the tip side than the axial position of the boundary b2 between the first site s1 and the second site s2. The description of the configuration of the medical device 1C that is common to the medical device 1 of the first embodiment is omitted.

The step portion 130C is provided at a position closer to the tip side than the boundary b2 between the first site s1 and the second site s2 and closer to the base end than to the tip end of the second site s2. In other words, the step portion 130C is provided closer to the base side than the center of the second site s2 in the axial direction of the second site s2. The maximum outer diameter portion 140C is provided at a position closer to the tip side than the boundary b2 between the first site s1 and the second site s2 and closer to the base end than to the tip end of the second site s2. In other words, the maximum outer diameter portion 140C is provided closer to the base side than the center of the second site s2 in the axial direction of the second site s2. With such a medical device 1C of the third embodiment, the distance between the outer periphery of the resin coating 120C and the inner periphery of the medical equipment such as a catheter is reduced, and the area of contact between the resin coating 120C and the inner periphery of the medical equipment is increased. Thus, the frictional resistance between the medical device 1C and the medical equipment can prevent the retraction of the medical device 1C when the medical device 1C encounters resistance from the blood vessel wall or the like.

### Fourth Embodiment

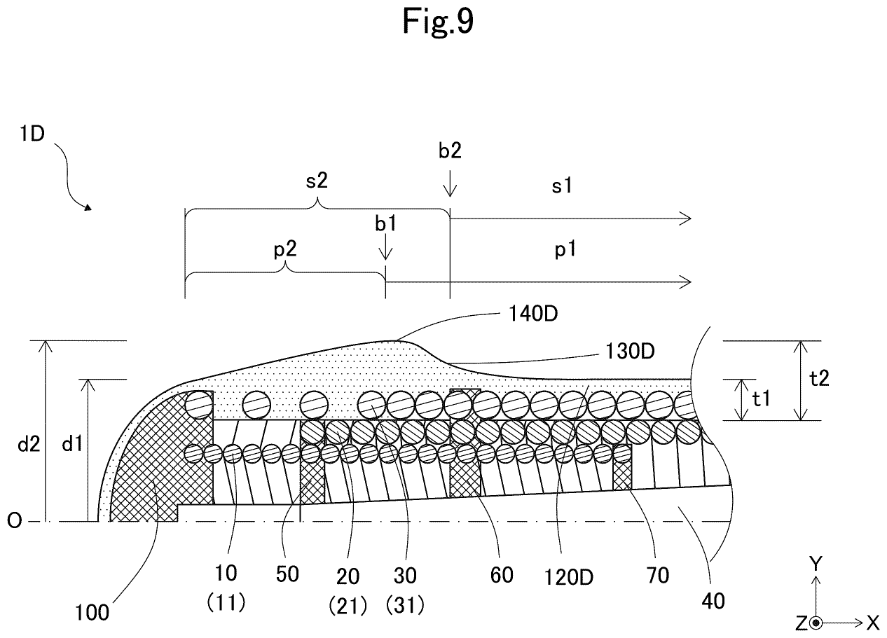

FIG. 9 is an explanatory diagram illustrating an example of an enlarged view of a resin coating 120D of a medical device 1D of a fourth embodiment. The medical device 1D is different from the medical device 1 of the first embodiment in that the step portion 130D does not have a surface substantially perpendicular to the axis of the medical device 1D, and an outer peripheral surface near the maximum outer diameter portion 140D is a curved surface. The description of the configuration of the medical device 1D that is common to the medical device 1 of the first embodiment is omitted.

The step portion 130D has a tapered shape in which the outer diameter gradually decreases toward the base side of the medical device 1D. The outer peripheral surface near the maximum outer diameter portion 140D is a curved surface, and in the axial direction of the resin coating 120, the outer diameter of the resin coating 120 closer to the tip side than the maximum outer diameter portion 140D gradually decreases toward the tip side. In the axial direction of the resin coating 120, the outer diameter of the resin coating 120 closer to the base side than the maximum outer diameter portion 140D gradually decreases toward the base side. With such a medical device 1D of the fourth embodiment, the distance between the outer periphery of the resin coating 120D and the inner periphery of the medical equipment such as a catheter is reduced, and the area of contact between the resin coating 120D and the inner periphery of the medical equipment is increased. Thus, the frictional resistance between the medical device 1D and the medical equipment can prevent the retraction of the medical device 1D when the medical device 1D encounters resistance from the blood vessel wall or the like.

### Fifth Embodiment

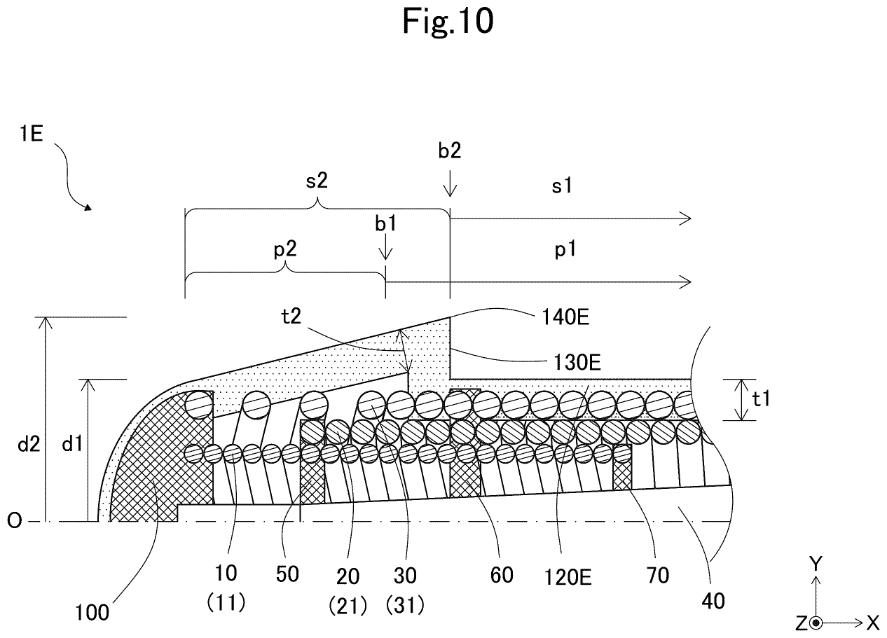

FIG. 10 is an explanatory diagram illustrating an example of an enlarged view of a resin coating 120E of a medical device 1E of a fifth embodiment. The medical device 1E is different from the medical device 1 of the first embodiment in that the film thickness t1 of the resin coating 120E at the first site s1 is the same as the film thickness t2 of the resin coating 120E at the second site s2. The description of the configuration of the medical device 1E that is common to the medical device 1 of the first embodiment is omitted.

In the resin coating 120E, the representative outer diameter rd2 of the resin coating 120E at the second site s2 is larger than the representative outer diameter rd1 of the resin coating 120E at the first site s1, while the representative film thickness rt2 of the resin coating 120E at the second site s2 is the same as the representative film thickness rt1 of the resin coating 120E at the first site s1. In other words, the representative film thicknesses (rt1, rt2) of the resin coating 120E are substantially constant in the axial direction of the resin coating 120E. However, the resin coating 120E protrudes radially outward of the medical device 1E at the second site s2 so as to form a maximum outer diameter portion 140E and a step portion 130E. With such a medical device 1E of the fifth embodiment, the distance between the outer periphery of the resin coating 120E and the inner periphery of the medical equipment such as a catheter is reduced, and the area of contact between the resin coating 120E and the inner periphery of the medical equipment is increased. Thus, the frictional resistance between the medical device 1E and the medical equipment can prevent the retraction of the medical device 1E when the medical device 1E encounters resistance from the blood vessel wall or the like.

### Sixth Embodiment

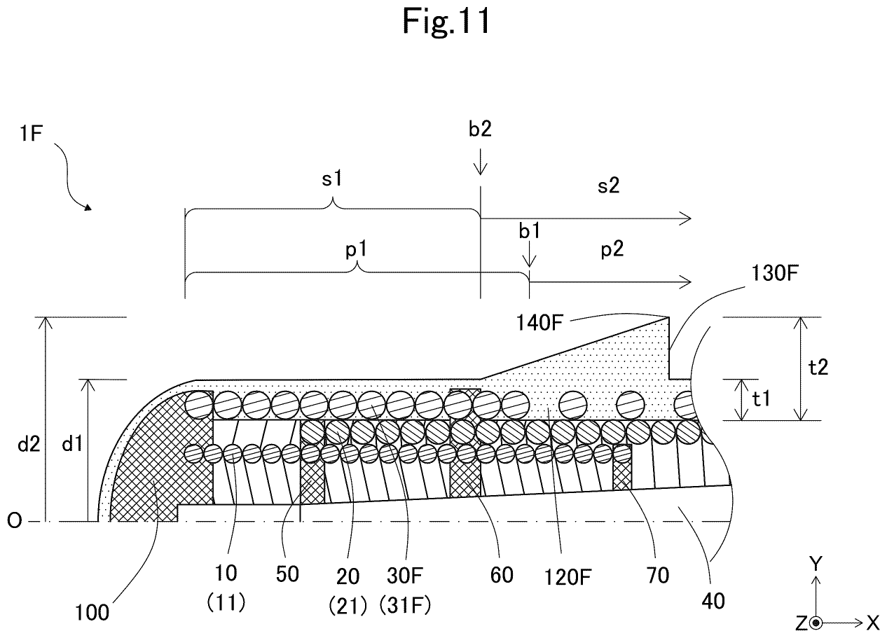

FIG. 11 is an explanatory diagram illustrating an example of an enlarged view of a resin coating 120F of a medical device 1F of a sixth embodiment. The medical device 1F is different from the medical device 1 of the first embodiment in that the second site s2 is disposed closer to the base side than the first site s1 in the axial direction of the medical device 1F. The description of the configuration of the medical device 1F that is common to the medical device 1 of the first embodiment is omitted.

A wire 31F near the tip end of a third coil body 30F is set to the first pitch, and the wire 31F closer to the base side than the wire 31F set to the first pitch is set to the second pitch. That is, the second pitch section p2 is disposed closer to the base side than the first pitch section p1. As a result, the second site s2 is also disposed closer to the base side than the first site s1. A step portion 130F and a maximum outer diameter portion 140F are disposed closer to the base side than the boundary b2 between the first site s1 and the second site s2. With such a medical device 1F of the sixth embodiment, the distance between the outer periphery of the resin coating 120F and the inner periphery of the medical equipment such as a catheter is reduced, and the area of contact between the resin coating 120E and the inner periphery of the medical equipment is increased. Thus, the frictional resistance between the medical device 1F and the medical equipment can prevent the retraction of the medical device 1F when the medical device 1F encounters resistance from the blood vessel wall or the like.

### Seventh Embodiment

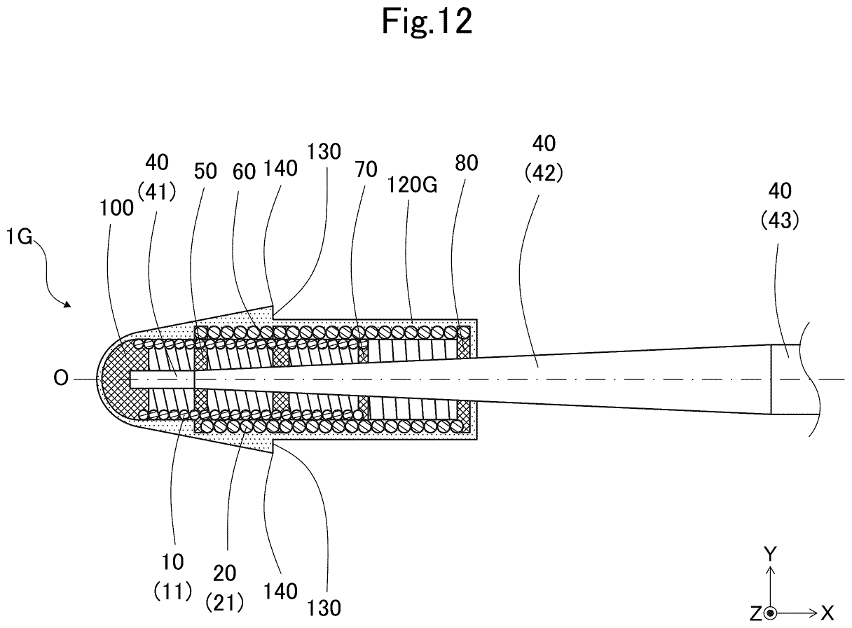

FIG. 12 is an explanatory diagram illustrating an example of a tip portion of a medical device 1G of a seventh embodiment. The medical device 1G is different from the medical device 1 of the first embodiment in that the medical device 1G does not include the third coil body 30. The description of the configuration of the medical device 1G that is common to the medical device 1 of the first embodiment is omitted.

The medical device 1G does not include the third coil body 30, but includes the first coil body 10 and the second coil body 20. A resin coating 120G covers the outer periphery of the second coil body 20. With the medical device 1G of the seventh embodiment, the distance between the outer periphery of the resin coating 120G and the inner periphery of the medical equipment such as a catheter is reduced, and the area of contact between the resin coating 120G and the inner periphery of the medical equipment is increased. Thus, the frictional resistance between the medical device 1G and the medical equipment can prevent the retraction of the medical device 1G when the medical device 1G encounters resistance from the blood vessel wall or the like.

### <Modifications>

The scope of the disclosure is not intended to be limited to the above embodiments and can be implemented in various aspects without departing from the spirit of the disclosure. For example, the following modifications are possible.

### <Modification 1>

The axial lengths of the straight portion 41, the tapered portion 42, and the large diameter portion 43 can be freely determined. For example, in the first to seventh embodiments, the axial length of the straight portion 41 is smaller than the axial length of the tapered portion 42. However, the axial length of the straight portion 41 may be larger than the axial length of the tapered portion 42. Further, the cross-sectional shapes of the straight portion 41, the tapered portion 42, and the large diameter portion 43 can be freely determined. For example, in the first embodiment, the cross-sectional shapes of the straight portion 41, the tapered portion 42, and the large diameter portion 43 are circular shapes. However, the cross-sectional shapes of the straight portion 41, the tapered portion 42, and the large diameter portion 43 may be elliptical shapes or polygonal shapes such as triangular and rectangular shapes.

### <Modification 2>

The medical devices (1, 1B, 1C, 1D, 1E, 1F, 1G) do not need to include three layers of coil bodies as exemplified in the seventh embodiment. The medical devices (1, 1B, 1C, 1D, 1E, 1F, 1G) may be configured to include only the first coil body 10, and the resin coating 120 may cover the outer periphery of the first coil body 10.

This disclosure has been made to solve at least part of the problems described above and can be achieved in the following aspects.

(2) The medical device of the above aspect may include a fixing portion that fixes coils of a wire of the outer layer coil at a boundary between the first site and the second site.

According to this configuration, the boundary between the first site and the second site can be easily set by the fixing portion.

(3) In the medical device of the above aspect, the resin coating may include a step portion on an outer peripheral surface of the resin coating at the boundary between the first site and the second site, the step portion having an outer diameter of the resin coating decreasing from the second site to the first site.

According to this configuration, the step portion can further prevent the retraction of the medical device when the medical device encounters resistance from the blood vessel wall or the like.

(4) In the medical device of the above aspect, in the resin coating, a maximum outer diameter portion having the largest outer diameter of the resin coating is formed in the second site, the maximum outer diameter portion being formed at a position closer to a base end than to a tip end of the second site.

According to this configuration, it is possible to prevent the retraction of the medical device when the medical device encounters resistance from the blood vessel wall or the like, while keeping the outer diameter of a tip portion of the medical device small.

(5) In the medical device of the above aspect, the representative outer diameter of the resin coating may be a maximum outer diameter of the resin coating.

According to this configuration, the maximum outer diameter of the resin coating covering the outer periphery of the second site is larger than the maximum outer diameter of the resin coating covering the outer periphery of the first site, thereby making it possible to prevent the retraction of the medical device when the medical device encounters resistance from the blood vessel wall or the like.

(6) In the medical device of the above aspect, the representative outer diameter of the resin coating may be an average outer diameter of the resin coating.

According to this configuration, the average outer diameter of the resin coating covering the outer periphery of the second site is larger than the average outer diameter of the resin coating covering the outer periphery of the first site, thereby making it possible to prevent the retraction of the medical device when the medical device encounters resistance from the blood vessel wall or the like.

(7) In the medical device of the above aspect, in the resin coating, a representative film thickness of the resin coating covering the outer periphery of the second site may be larger than the representative film thickness of the resin coating covering the outer periphery of the first site.

According to this configuration, the representative film thickness of the resin coating covering the outer periphery of the second site is larger than the representative film thickness of the resin coating covering the outer periphery of the first site, thereby making it possible to prevent the retraction of the medical device when the medical device encounters resistance from the blood vessel wall or the like.

(8) One aspect of the disclosed embodiments is a medical device including: an outer layer coil having a first site that includes a section in which a coil pitch is a first pitch and a second site that includes the section in which the coil pitch is a second pitch that is wider than the first pitch; and a resin coating that covers the outer layer coil, a representative film thickness of the resin coating covering an outer periphery of the second site being larger than the representative film thickness of the resin coating covering the outer periphery of the first site.

According to this configuration, the representative film thickness of the resin coating covering the outer periphery of the second site is larger than the representative film thickness of the resin coating covering the outer periphery of the first site, thereby making it possible to prevent the retraction of the medical device when the medical device encounters resistance from the blood vessel wall or the like.

(9) In the medical device of the above aspect, the second site may include the section having the first pitch.

(10) One aspect of the disclosed embodiments is a method for producing a medical device, the method including steps of: preparing an outer layer coil having a first site that includes a section in which a coil pitch is a first pitch and a second site that includes the section in which the coil pitch is a second pitch that is wider than the first pitch; and applying a resin coating to the prepared outer layer coil along an axial direction of the outer layer coil such that a representative outer diameter of the resin coating covering an outer periphery of the second site is made larger than the representative outer diameter of the resin coating covering the outer periphery of the first site.

According to this production method, the medical device can be easily produced in which the representative outer diameter of the resin coating covering the outer periphery of the second site is larger than the representative outer diameter of the resin coating covering the outer periphery of the first site.

Note that the disclosed embodiments can be achieved in various aspects. For example, the disclosed embodiments can be achieved in a form of a guidewire, a method for producing a guidewire, a method for producing a catheter, an endoscope, a dilator, or the like.

### DESCRIPTION OF REFERENCE NUMERALS

1 medical device
10 first coil body
11 wire of first coil body
20 second coil body
21 wire of second coil body
30 third coil body
31 wire of third coil body
40 core shaft
41 straight portion
42 tapered portion
43 large diameter portion
50 first fixing portion
60 second fixing portion
70 third fixing portion
80 fourth fixing portion
100 tip side fixing portion
110 base side fixing portion
120 resin coating
130 step portion
140 maximum outer diameter portion
p1 first pitch section
p2 second pitch section
s1 first site
s2 second site
b1 boundary between first pitch section and second pitch section
b2 boundary between first site and second site
d1 outer diameter of first site
d2 outer diameter of second site
t1 film thickness of first site
t2 film thickness of second site

What is claimed is:

1. A medical device comprising:
an outer layer coil having a first site that includes a section in which a coil pitch is a first pitch and a second site that includes a section in which the coil pitch is a second pitch, and the second pitch is wider than the first pitch; and
a resin coating that covers the outer layer coil, a representative outer diameter of the resin coating covering an outer periphery of the second site being larger than a representative outer diameter of the resin coating covering an outer periphery of the first site.

2. The medical device according to claim 1, further comprising a fixing portion that fixes coils of a wire of the outer layer coil at a boundary between the first site and the second site.

3. The medical device according to claim 1, wherein the resin coating includes a step portion on an outer peripheral surface of the resin coating at the boundary between the first site and the second site, the step portion having an outer diameter of the resin coating decreasing from the second site to the first site.

4. The medical device according to claim 1, wherein, in the resin coating, a maximum outer diameter portion having the largest outer diameter of the resin coating is formed in the second site, the maximum outer diameter portion being formed at a position closer to a base end than to a tip end of the second site.

5. The medical device according to claim 1, wherein the representative outer diameter of the resin coating is a maximum outer diameter of the resin coating.

6. The medical device according to claim 1, wherein the representative outer diameter of the resin coating is an average outer diameter of the resin coating.

7. The medical device according to claim 1, wherein, in the resin coating, a representative film thickness of the resin coating covering the outer periphery of the second site is larger than the representative film thickness of the resin coating covering the outer periphery of the first site.

8. The medical device according to claim 2, wherein the resin coating includes a step portion on an outer peripheral surface of the resin coating at the boundary between the first site and the second site, the step portion having an outer diameter of the resin coating decreasing from the second site to the first site.

9. The medical device according to claim 2, wherein, in the resin coating, a maximum outer diameter portion having the largest outer diameter of the resin coating is formed in the second site, the maximum outer diameter portion being formed at a position closer to a base end than to a tip end of the second site.

10. The medical device according to claim 2, wherein the representative outer diameter of the resin coating is a maximum outer diameter of the resin coating.

11. The medical device according to claim 2, wherein the representative outer diameter of the resin coating is an average outer diameter of the resin coating.

12. The medical device according to claim 2, wherein, in the resin coating, a representative film thickness of the resin coating covering the outer periphery of the second site is larger than the representative film thickness of the resin coating covering the outer periphery of the first site.

13. The medical device according to claim 3, wherein, in the resin coating, a maximum outer diameter portion having the largest outer diameter of the resin coating is formed in the second site, the maximum outer diameter portion being formed at a position closer to a base end than to a tip end of the second site.

14. The medical device according to claim 3, wherein, in the resin coating, a representative film thickness of the resin coating covering the outer periphery of the second site is larger than the representative film thickness of the resin coating covering the outer periphery of the first site.

15. A medical device comprising:
an outer layer coil having a first site that includes a section in which a coil pitch is a first pitch and a second site that includes a section in which the coil pitch is a second pitch, and the second pitch is wider than the first pitch; and
a resin coating that covers the outer layer coil, a representative film thickness of the resin coating covering an outer periphery of the second site being larger than a representative film thickness of the resin coating covering an outer periphery of the first site.

16. The medical device according to claim 1, wherein the second site includes the section having the first pitch.

17. The medical device according to claim 15, wherein the second site includes the section having the first pitch.

18. A method for producing a medical device, comprising steps of:
preparing an outer layer coil having a first site that includes a section in which a coil pitch is a first pitch and a second site that includes a section in which the coil pitch is a second pitch, and the second pitch is wider than the first pitch; and
applying a resin coating to the prepared outer layer coil along an axial direction of the outer layer coil such that a representative outer diameter of the resin coating covering an outer periphery of the second site is made larger than a representative outer diameter of the resin coating covering an outer periphery of the first site.

19. The medical device according to claim 1, wherein the second site is disposed closer to a tip side of the outer layer coil than the first site.

20. The medical device according to claim 15, wherein the second site is disposed closer to a tip side of the outer layer coil than the first site.
